SEQUENCE LISTING (40 sequences)

SEQ ID NO: 1 - DNA, 2427 bp, Pan troglodytes
gtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctccaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttgagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattaaaagcgtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccaaagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcggcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctga

SEQ ID NO: 2 - DNA, 2427 bp, Pan troglodytes; identical to SEQ ID NO: 1
  CDS (1)..(2427)

SEQ ID NO: 3 - protein, 808 aa, Pan troglodytes
VVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIKSVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRRQVELYRLLSRNTYLEWEDSVLGRHIFWRRLRKALLDGKSWNPEGTVGTGCNWQEATSI

SEQ ID NO: 4 - DNA, 2427 bp, Gorilla gorilla
gtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctccaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttattcaatctttcaaattacctgagtatttttctaatctgaccaatctagagtacttggacctttccagcaacaagattcaaagtatttattgcacagacttgcgggttctacatcaaatgcccctactcaatctctctttagacctgtccctgaaccctatgacctttatccaaccaggtgcatttaaagaaattaggcttcataagctgactttgagaaataattttgatagtttaaatgtaatgaaaacttgtattcaaggtctggctggtttagaagtccgtcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcggaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccgggttcttgattacagtctcaatcacataatgacttccaaaaaacaggaactacagcattttccaagcagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacgtctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagtcgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctga

SEQ ID NO: 5 - DNA, 2427 bp, Gorilla gorilla; identical to SEQ ID NO: 4
  CDS (1)..(2427)

SEQ ID NO: 6 - protein, 808 aa, Gorilla gorilla
VVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEYLDLSSNKIQSIYCTDLRVLHQMPLLNLSLDLSLNPMTFIQPGAFKEIRLHKLTLRNNFDSLNVMKTCIQGLAGLEVRRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLRVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENVYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 7 - DNA, 2406 bp, Hylobates lar
gtggttcctaacattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagcctcctctccaccttaatattgacaggaaaccccatccagagtttagctctgggagccttttctggactatcaagtttacagaagctagtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcaaagacttgcaggttctacatcaaatgcccctactcaatctctctttagacctgtccctgaaccctatgaactttatccaaccaggtgcatttaaagaaattagccttcrtaagctgactttaagaaataattttgatagtttaaatgtaatgaaaacttgtattcaaggtctggctggtttagaagtccatcgtttggttctgggagaatttagaaatgaaggaaacttggaagagtttgacaaatctgctctagagggcctgtgcaatttgaccattgaagaattccgattagcatacttagaccactacctcgatgatattattgacttatttaattgtttggcaaatgtttcttcattttccctggtgagtgtgactattaaaagggtagaagacttttcttataatttcggatggcaacatttagaattagttaactgtaaatttggacagtttcccacattgaacctcaaatctctcaaaaggcttactttcactgccaacagaggtgggaatgctttttcagaagttgatctaccaagccttgagtttctagatctcagtagaaatggcttgagtttcaaaggttgctgttctcaaagtgattttgggacaaacagcctaaagtatttagatctgagcttcaatgatgttattaccatgagttcaaacttcttgggcttagaacagctagaacatctggatttgcagcattccaatttgaaacaaatgagtgaattttcagtattcctatcactcagaaacctcatttaccttgacatttctcatactcacaccagagttgctttcaatggcatcttcaatggcttgtccaatctcgaagtcttgaaaatggctggcaattctttccaggaaaacttccttccagatatcttcacagagctgagaaacttgaccttcctggacctctctcagtgtcaactggagcaattgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcgttttccaagtagtctagccttcttaaatcttactcagaatgactttgcttgtacttgtgaacacgagagtttcctgcagtggatcaaggaccagaggcagctcttggtggaagttgaacgaatggaatgtgcaacaccttcagataagcagggcatgcctgtgctgagtttgaatatcacctgtcagatgaataagaccatcattggtgtgtcagtcctcagtgtgcttgtagtatctgttgtagcagttctggtctataagttctattttcacctgatgcttcttgctggctgcatgaagtatggtagaggtgaaaacacctatgatgcctttgttatctactccagccaggatgaggactgggtaaggaatgagctagtaaagaatttagaagaaggggtgcctccctttcagctctgccttcactacagagactttattccyggtgtggccattgctgccaacatcatccatgaaggtttccataaaagccgaaaggtgattgttgtggtgtcccagcacttcatccagagccgctggtgtatctttgagtatgagattgctcagacctggcagtttctgagcagtcatgctgggatcatcttcattgtcctgcagaaggtggagaagaccctgctcaggcagcaggtggagctgtaccgccttctcagcaggaacacttacctggagtgggaggatagtgtcctggggcggcacattttctggagacgactcagaaaagccctgctggatggtaaatcatggaatccagaaggaacagtgggtacaggatgcaattag

SEQ ID NO: 8 - DNA, 2406 bp, Hylobates lar; identical to SEQ ID NO: 7
  CDS (1)..(2406)

SEQ ID NO: 9 - protein, 801 aa, Hylobates lar
  misc_feature (198)..(198): The ′Xaa′ at location 198 stands for Arg, or His.
VVPNITYQCMELNFYKIPDNLPFSTKNLDLSFNPLRHLGSYSFFSFPELQVLDLSRCEIQTIEDGAYQSLSLLSTLILTGNPIQSLALGAFSGLSSLQKLVAVETNLASLENFPIGHLKTLKELNVAHNLIQSFKLPEYFSNLTNLEHLDLSSNKIQSIYCKDLQVLHQMPLLNLSLDLSLNPMNFIQPGAFKEISLXKLTLRNNFDSLNVMKTCIQGLAGLEVHRLVLGEFRNEGNLEEFDKSALEGLCNLTIEEFRLAYLDHYLDDIIDLFNCLANVSSFSLVSVTIKRVEDFSYNFGWQHLELVNCKFGQFPTLNLKSLKRLTFTANRGGNAFSEVDLPSLEFLDLSRNGLSFKGCCSQSDFGTNSLKYLDLSFNDVITMSSNFLGLEQLEHLDLQHSNLKQMSEFSVFLSLRNLIYLDISHTHTRVAFNGIFNGLSNLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQRFPSSLAFLNLTQNDFACTCEHESFLQWIKDQRQLLVEVERMECATPSDKQGMPVLSLNITCQMNKTIIGVSVLSVLVVSVVAVLVYKFYFHLMLLAGCMKYGRGENTYDAFVIYSSQDEDWVRNELVKNLEEGVPPFQLCLHYRDFIXGVAIAANIIHEGFHKSRKVIVVVSQHFIQSRWCIFEYEIAQTWQFLSSHAGIIFIVLQKVEKTLLRQQVELYRLLSRNTYLEWEDSVLGRHIFWRRLRKALLDGKSWNPEGTVGTGCN

SEQ ID NO: 10 - DNA, 2388 bp, Macaca mulatta
gtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctccactttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccagtctttcaaattacctgagtatttttctaatctgaccaatctagagcacttggacctttccagtaacaagattcaaaatatttattgcaaagacttgcaggttctacatcaaatgcccctatccaatctctctttagacctgtccctgaaccctataaactttatccaaccaggtgcatttaaagaaattaggcttcataagctgactttgagaagtaattttgatgatttaaatgtaatgaaaacttgtattcaaggtctggctggtttagaagtccatcgtttggttctgggagaatttagaaatgaaagaaacttggaagagtttgacaaatcttctctggagggattgtgcaatttgaccattgaagaattccgattaacatacttagactactacctcgataatattattgacttatttaattgtttggcaaatgtttcttcattttccctggtgagtgtgagtattaaaagggtagaagacttttcttataatttcagatggcaacatttagaattagttaactgtaaatttgaacagtttcccacattggaactcgaatctctcaaaaggcttactttcactgccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatgatgttattaccatgagttcaaacttcttgggcttagaaaaactagaacatctggatttccagcattccaatttgaaacagatgagtcaattttcagtattcctatcactcagaaacctcatttaccttgacatttctcatactcacaccagagttgctttcaatggcatcttcgatggcttgctcagtctcaaagtcttaaaaatggctggcaattctttccaggaaaacttccttccagatatcttcacagatctgaaaaacttgaccttcctggacctctctcagtgtcaattggagcagttgtctccaacagcatttgacacactcaacaagcttcaggtactaaatatgagccacaacaacttcttttcattggatacgtttccttataagtgtctgccctccctccaggttctcgattacagtctcaatcacataatgacttccaacaaccaggaactacagcattttccaagtagtctagctttcttaaatcttactcagaatgactttgcttgtacttgtgaacaccagagtttcctgcagtggatcaaggaccagaggcagctcttggtggaagctgaacgaatggaatgtgcaacaccttcagataaacagggcatgccggtgctgagtttgaatattacctgtcagatgaataagaccatcattggtgtgtctgtgttcagtgtgcttgtggtatctgttgtagcagttctggtctataagttctattttcacctgatgcttcttgctggctgcataaastatggtagaggtgaaaacatctatgatgcctttgttatctactcaagccaggatgaggactgggtaaggaatgaactagtaaagaatttagaagaaggggtgcctccctttcagctctgccttcactacagagactttattcccggtgtggccattgctgcaaacatcatccatgaaggtttccataaaagccgaaaggtgattgttgtggtgtcccagcacttcatccagagccgctggtgtatctttgaatatgagattgctcagacctggcagtttctgagcagtcgtgcaggcataatcttcattgtcctgcagaaggtggagaagaccctgctcaggcagcaggtggagctgtaccgccttctcagcaggaacacttacctggagtgggaggacagtgtcctggggcagcacatcttctggagacgactcagaaaagccctgttggatggcagatcgtggaatccagaagaacagtag

SEQ ID NO: 11 - DNA, 2388 bp, Macaca mulatta; identical to SEQ ID NO: 10
  CDS (1)..(2388)

SEQ ID NO: 12 - protein, 795 aa, Macaca mulatta
  misc_feature (635)..(635): The ′Xaa′ at location 635 stands for Lys, or Asn.
VVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQNIYCKDLQVLHQMPLSNLSLDLSLNPINFIQPGAFKEIRLHKLTLRSNFDDLNVMKTCIQGLAGLEVHRLVLGEFRNERNLEEFDKSSLEGLCNLTIEEFRLTYLDYYLDNIIDLFNCLANVSSFSLVSVSIKRVEDFSYNFRWQHLELVNCKFEQFPTLELESLKRLTFTANKGGNAFSEVDLPSLEFLDLSRNGLSFKGCCSQSDFGTTSLKYLDLSFNDVITMSSNFLGLEKLEHLDFQHSNLKQMSQFSVFLSLRNLIYLDISHTHTRVAFNGIFDGLLSLKVLKMAGNSFQENFLPDIFTDLKNLTFLDLSQCQLEQLSPTAFDTLNKLQVLNMSHNNFFSLDTFPYKCLPSLQVLDYSLNHIMTSNNQELQHFPSSLAFLNLTQNDFACTCEHQSFLQWIKDQRQLLVEAERMECATPSDKQGMPVLSLNITCQMNKTIIGVSVFSVLVVSVVAVLVYKFYFHLMLLAGCIXYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGQHIFWRRLRKALLDGRSWNPEEQ

SEQ ID NO: 13 - DNA, 2238 bp, Cebus capucinus
tgtgaaatccacacaattgaagatggtgcatatcagagcctaagccacctctccaccttaatattgacaggaaatcctatccagaatttagccctgggagccttttctggactatcaagtttacagaaactggtagctgtggagacacatctgttatcgctagaaagcttccccattggacatctcaaaactttgaaggaccttaatgtggctcacaatctaatccaatctttcaaattacctgagtatttttctaatctgaccaatctagagcacttggacctttctagtaacaatattcaaaatatttattgcaaagacttgcaggttctacatcaaatgcccctactcaatctctctttagacctgtccctgaaccctataaactttattcagccaggtgcatttaaagaaattaggctccgtaagctgactttgagaaataattttgatagtttaaatgtaatgaaaacttgcattcacggtctggctgggttagaagtccatcgtttggttctgggagaatttagaaatgaaagaaatattgaagactttgacaaatctgctctggagggcctgtgcaatttgaccatcaaagaattccgattagcatacttagacaactttccagatgatattattgacttatttaattgtttggtaaatgtttcttcattttccctgttgagtgtgtatattaaaagagtagaagacttttcttataatttcagatggcaacatttagaattagttaactgtatatttcaacagtttcctccactgaaactcaaatctctcaaaaggcttactttcagtaaaaacaaaggtaggaatcattttgcagaagttgatctgccaagccttgagtttctagatctcagtagaaatggcttgagtttcaaaggttgctgttctcaatctgattttgggacgaccagcctaaagtatttagatctgagcttcaatgatgttattaccatgagttcaaacttcttaggcttagaacaactagaacacttggatttccagcattccaatttgaaacaaatgagtgagttttcagtatttctatcactcagaaacctcatttaccttgacatttctcatactcacaccagagttgctttcaatggcatctttaatggcttgttcagtctcaaagtcttgaaaatggctggaaattctttccagcaaaacttccttgcagatatcttcacagatctgaataacttgatattcctggacctttctgagtgtcaactggagcagttgtctccaacagcatttgactcacttcccagacttcagatactaaatatgagccacaacaagttctttgcattggatacatttccttataagcatctctactccctccacgttctggattacagtctcaatcacatagggacttccaaaaatcaggaactacagcattttccaagtagtctagctttcttaaatcttactcaaaatgactttgcttgtacttgtgaacaccagagtttcctgcagtggatcaaggaccagaggcggctattggtggaagttgaacgaatggaatgcgcaacacctttaaataggaagggcatacctgtgctgagtttgaatatcacctgtcagatgagtaagaccatcattggtgtgtcagtgctcagtgtgcttgtggtatctgttgtagcagttctggtctataagttctattttcacctgatgcttcttgctggctgcataaagtatggtagaggtgaaaacacctatgatgcctttgttatctactcaagccaggatgaggactgggtaaggaatgaactagtaaagaatttagaagaaggggtgcctccttttcagctctgccttcactacagagactttattcccggtgtggccattgctgccaacatcatccatgaaggtttccataaaagccgaaaggtgattgttgtggtatcccagcacttcatccagagccgctggtgtatctttgaatatgagattgctcagacctggcagtttctgagcagtcgtgctggtatcatcttcattgtcctgcagaaggtggagaagtccctgctcaggcagcaggtggagctgtaccgccttctcagcaggaacacctacctggagtgggaggacagtgtcctggggaggcatatcttctggaggcgactcagaaaagccctgctgaatggtagaccgtggagtccagaaggaacagtgggtgcaggatgcgattag

SEQ ID NO: 14 - DNA, 2238 bp, Cebus capucinus; identical to SEQ ID NO: 13
  CDS (1)..(2238)

SEQ ID NO: 15 - protein, 745 aa, Cebus capucinus
CEIHTIEDGAYQSLSHLSTLILTGNPIQNLALGAFSGLSSLQKLVAVETHLLSLESFPIGHLKTLKDLNVAHNLIQSFKLPEYFSNLTNLEHLDLSSNNIQNIYCKDLQVLHQMPLLNLSLDLSLNPINFIQPGAFKEIRLRKLTLRNNFDSLNVMKTCIHGLAGLEVHRLVLGEFRNERNIEDFDKSALEGLCNLTIKEFRLAYLDNFPDDIIDLFNCLVNVSSFSLLSVYIKRVEDFSYNFRWQHLELVNCIFQQFPPLKLKSLKRLTFSKNKGRNHFAEVDLPSLEFLDLSRNGLSFKGCCSQSDFGTTSLKYLDLSFNDVITMSSNFLGLEQLEHLDFQHSNLKQMSEFSVFLSLRNLIYLDISHTHTRVAFNGIFNGLFSLKVLKMAGNSFQQNFLADIFTDLNNLIFLDLSECQLEQLSPTAFDSLPRLQILNMSHNKFFALDTFPYKHLYSLHVLDYSLNHIGTSKNQELQHFPSSLAFLNLTQNDFACTCEHQSFLQWIKDQRRLLVEVERMECATPLNRKGIPVLSLNITCQMSKTIIGVSVLSVLVVSVVAVLVYKFYFHLMLLAGCIKYGRGENTYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKSLLRQQVELYRLLSRNTYLEWEDSVLGRHIFWRRLRKALLNGRPWSPEGTVGAGCD

SEQ ID NO: 16 - DNA, 2406 bp, Saimiri sciureus
gtggttcctaacgttacttatcaatgcatggaactgaatytctacaaaatccccgacaacatccccttctcaactaagaacctggacctgagctttaaccccctgaggcatttaggcagccatagcttcttcaatttcccagaactgcaggtgctggatttatccaggtgtgacatccagacaatcgaagatggggcatatcagagcctaagccacctctccaccttaatattgacaggaaatcctatccagaatttagccctgggagccttttctggactatcaagtttacagaagctggtggctgtggagacacatctgttatcactagagaacttccccattggacatctcaaaactttgaaggaccttaatgtggctcacaatctaatccaatctttcaaattacctgagtatttttctaatctgaccaatctagagcacttggacctttctagtaacaatattcaaaatatttattgcaaagacttgcaggttctacatcaaatgcccctactcaatctctctttagacctgtccctgaaccctataaactttattcaaccaggtgcgtttaaagaaattaggctccataagctgactttgagaaataattttgatagtttaaatgcaatgaaaacttgcattcaaggtctggctgggttagaagtccatcgtttggttctgggagaatttagaaatgaaagaaatattgaagactttgacaaatctgctctggagggcctgtgcaatttgaccattaatgaattccgattagcttacttagatgactttctagatgatattattgacttatttaactgtttagcaaatgtttcttcattttccctggtgaatgtgcatattaaaagagtagaagacttttcttataattttagatggcaacatttagaattagttaactgtgtatttcaacagtttcctccactgaaactcaaatctctcaaaaggcttactttcactgccaacaaaggtaggaatcatttttcagaagttgatcttccaagccttgagtttctagatctcagtagaaatggcttgagtttcaaaggttgctgttctcaatctgattttgggacgaccagcctaaagtatttagatctgagcttcaatgacgttattaccatgggttcaaacttcttaggcttagaacaactagaacacttggatttccagcattccaatttgaaacaaatgagtgagttttcagtattcctatcactcagaaacctcatttaccttgacatttctcatactcacaccagagttgctttcaatggcatctttaatggcttgttcagtctcaaagtcttgaaaatggctggaaattctttccagcaaaacttccttgaagatatcttcacrgatctgaataacttgatattcctggacctctctgagtgtcagctggagcagttgtctccaacagcatttgactcacttcccagacttcggatactaaatatgagccacaacaacttctttgcattggatacattcccttacaagcatctctactccctccaggttctggattacagtctcaatcatatagggacttccaaaaatcaggaactgcagcattttccaagtagtctagctttcttaaatcttactcaaaatgactttgcttgtacttgtgaacaccagagtttcctgcagtggatcaaggaccagaggcggctgttggtggaagttgaacaaatggaatgtgcaacacctttaaataggaagggcatacctgtgctgagtttgaatatcacctgtcagatgagtaagactatcattggtgtgtcagtgctcagtgtgcttgtggtatctgttgtagcagttctggtctataagttctattttcacctgatgcttcttgctggctgcataaagtatggtagaggtgaaaacacctatgatgcctttgttatctactcaagccaggatgaggactgggtaaggaatgaactagtaaagaatttagaagaaggggtgcctccctttcagctctgccttcactacagagactttattcccggtgtggccattgctgccaacatcatccatgaaggtttccataaaagccgaaaggtgattgttgtggtatctcagcacttcatccagagccgctggtgtatctttgaatatgagattgctcagacctggcagtttctgagcagtcgtgctggtatcatcttcattgtcctgcagaaggtggagaagtccctgctcaggcagcaggtggagctgtaccgccttctcagcaggaacacttacctggagtgggaggacagtgtcctggggaggcacatcttctggagacgactcagaaaagccctgctggatggtagaccgtggaatccagaaggaacagtgggtgcaggatgcgaatag

SEQ ID NO: 17 - DNA, 2406 bp, Saimiri sciureus; identical to SEQ ID NO: 16
  CDS (1)..(2406)

SEQ ID NO: 18 - protein, 801 aa, Saimiri sciureus
  misc_feature (14)..(14): The ′Xaa′ at location 14 stands for Leu, or Phe.
VVPNVTYQCMELNXYKIPDNIPFSTKNLDLSFNPLRHLGSHSFFNFPELQVLDLSRCDIQTIEDGAYQSLSHLSTLILTGNPIQNLALGAFSGLSSLQKLVAVETHLLSLENFPIGHLKTLKDLNVAHNLIQSFKLPEYFSNLTNLEHLDLSSNNIQNIYCKDLQVLHQMPLLNLSLDLSLNPINFIQPGAFKEIRLHKLTLRNNFDSLNAMKTCIQGLAGLEVHRLVLGEFRNERNIEDFDKSALEGLCNLTINEFRLAYLDDFLDDIIDLFNCLANVSSFSLVNVHIKRVEDFSYNFRWQHLELVNCVFQQFPPLKLKSLKRLTFTANKGRNHFSEVDLPSLEFLDLSRNGLSFKGCCSQSDFGTTSLKYLDLSFNDVITMGSNFLGLEQLEHLDFQHSNLKQMSEFSVFLSLRNLIYLDISHTHTRVAFNGIFNGLFSLKVLKMAGNSFQQNFLEDIFXDLNNLIFLDLSECQLEQLSPTAFDSLPRLRILNMSHNNFFALDTFPYKHLYSLQVLDYSLNHIGTSKNQELQHFPSSLAFLNLTQNDFACTCEHQSFLQWIKDQRRLLVEVEQMECATPLNRKGIPVLSLNITCQMSKTIIGVSVLSVLVVSVVAVLVYKFYFHLMLLAGCIKYGRGENTYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKSLLRQQVELYRLLSRNTYLEWEDSVLGRHIFWRRLRKALLDGRPWNPEGTVGAGCE

SEQ ID NO: 19 - DNA, 2388 bp, Papio hamadryas
gtggttcctaacattacttatcaatgcatggagctgaatttctacaaaatccccgacaacatccccttctcaaccaagaacctggacctgagctttaatcccctgaggcatttaggcagctatagcttcctccgttttccagaactgcaggtgctggatttatccaggtgtgaaatccagacaattgaagatggggcatatcagagcctaagccacctctccaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccagtctttcaaattacctgagtatttttctaatctgaccaatctagagcacttggacctttccagtaacaagattcaaaatatttattgcaaagacttgcaggttctacatcaaatgcccctacccaatctctctttagacctgtccctgaaccctataaactttatccaaccaggtgcatttaaagaaattaggcttcataagctgactttgagaagtaattttgatgatttaaatgtaatgaaaacttgtattcaaggtctggctggtttagaagtccatcgtttggttctgggagaatttagaaatgaaagaaacttggaagagtttgacaaatctgctctggagggattgtgcaatttgaccattgaagaattccgattaacatacttagactactacctcgataatattattgacttatttaattgtttggcaaatgcttcttcattttccctggtgagtgtgaatattaaaagggtagaagacttttcttataatttcagatggcaacatttagaattagttaactgtaaatttgaacagtttcccacattggaactcgaatctctcaaaaggcttactttcactgccaacaaaggtgggaatgccttttcagaagttgatctaccaagccttgagtttctagatctcagtagaaatggcttgagtttcaaaggttgctgttctcaaagtgattttgggacaaccagcctaaagtatttagatctgagcttcaatgatgttattaccatgggttcaaacttcttgggcttagaacaactagaacatctggatttccagcattccaatttgaaacagatgagtcaattttcagtattcctatcactcagaaacctcatttaccttgacatttctcatactcacaccacagttgctttcaatggcattttcgatggcttgctcagtctcaaagtcttaaaaatggctggcaattctttccaggaaaacttccttccagatatcttcacagatctgaaaaacttgaccttcctggacctctctcagtgtcaactggagcagttgtctccaacagcatttgacacactcaacaagcttcaggtactaaatatgagccacaacaacttcttttcattggatgtgtttccttataagtgtctgccctccctccaggttctcgattacagtctcaatcacataatgacttccaaaaaccaggaacctcagcattttccaagtagtctagctttcttaaatcttactcagaatgactttgcttgtacttgtgaacaccagagtttcctgcagtggatcaaggaccagaggcagctcttggtggaagctgaacgaatggaatgtgcaacaccttcagataaacagggcatgcctgtgctgagtgtgaatattacctgtcagatgaataagaccatcattggtgtgtctgtgttcagtgtgcttgtggtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccctttcagctctgccttcactacagagactttattcccggtgtggccattgctgcaaacatcatccatgaaggtttccataaaagccgaaaggtgattgttgtggtgtcccagcacttcatccagagccgctggtgtatctttgaatatgagattgctcagacctggcagtttctgagcagtcgtgcaggcataatcttcattgtcctgcagaaggtggagaagaccctgctcaggcagcaggtggagctgtaccgccttctcagcaggaacacttacctggagtgggaggacagtgtcctagggcagcacatcttctggagacgactcagaaaagccctgttggatggcagatcgtggaatccagaagaacagtag

SEQ ID NO: 20 - DNA, 2388 bp, Papio hamadryas; identical to SEQ ID NO: 19
  CDS (1)..(2388)

SEQ ID NO: 21 - protein, 795 aa, Papio hamadryas
VVPNITYQCMELNFYKIPDNIPFSTKNLDLSFNPLRHLGSYSFLRFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQNIYCKDLQVLHQMPLPNLSLDLSLNPINFIQPGAFKEIRLHKLTLRSNFDDLNVMKTCIQGLAGLEVHRLVLGEFRNERNLEEFDKSALEGLCNLTIEEFRLTYLDYYLDNIIDLFNCLANASSFSLVSVNIKRVEDFSYNFRWQHLELVNCKFEQFPTLELESLKRLTFTANKGGNAFSEVDLPSLEFLDLSRNGLSFKGCCSQSDFGTTSLKYLDLSFNDVITMGSNFLGLEQLEHLDFQHSNLKQMSQFSVFLSLRNLIYLDISHTHTTVAFNGIFDGLLSLKVLKMAGNSFQENFLPDIFTDLKNLTFLDLSQCQLEQLSPTAFDTLNKLQVLNMSHNNFFSLDVFPYKCLPSLQVLDYSLNHIMTSKNQEPQHFPSSLAFLNLTQNDFACTCEHQSFLQWIKDQRQLLVEAERMECATPSDKQGMPVLSVNITCQMNKTIIGVSVFSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGQHIFWRRLRKALLDGRSWNPEEQ

SEQ ID NO: 22 - DNA, 2427 bp, Pan troglodytes
gtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctccaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttgagaaataattttgatagtttaaatgtaatgaaaacttgtattcaaggtctggctggtttagaagtccatcgtttggttctgggagaatttagaaatgaagaaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattaaaagcgtaaaagacttttcttataatttcggatggcaacatttagaattagttaagtgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccaaagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacgtggcagtttctgagcagtcgtgctggtatcatcttcattgtcctgcagaaggtggagaagaccctgctcaggcggcaggtggagctgtaccgccttctyagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctga

SEQ ID NO: 23 - DNA, 2427 bp, Pan troglodytes; identical to SEQ ID NO: 22
  CDS (1)..(2427)

SEQ ID NO: 24 - protein, 808 aa, Pan troglodytes
  misc_feature (758)..(758): The ′Xaa′ at location 758 stands for Leu.
VVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEENLEKFDKSALEGLCNLTIEEFRLAYLDYYLDDIIDLFNCLTNVSSFSLVSVTIKSVKDFSYNFGWQHLELVKCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRRQVELYRLXSRNTYLEWEDSVLGRHIFWRRLRKALLDGKSWNPEGTVGTGCNWQEATSI

SEQ ID NO: 25 - protein, 35 aa, Amino acid
CNLTIEEFRLTYLDYYLDDIIDLFNCLANASSFSL

SEQ ID NO: 26 - protein, 35 aa, Amino acid
CNLTIEEFRLTYLDYYLDGIIDLFNCLANASSFSL

SEQ ID NO: 27 - protein, 35 aa, Amino acid; identical to SEQ ID NO: 25

SEQ ID NO: 28 - protein, 35 aa, Amino acid; identical to SEQ ID NO: 25

SEQ ID NO: 29 - protein, 35 aa, Amino acid; identical to SEQ ID NO: 25

SEQ ID NO: 30 - protein, 35 aa, Amino acid
CNLTIEEFRLAYLDYYLDDIIDLFNCLANVSSFSL

SEQ ID NO: 31 - protein, 35 aa, Amino acid; identical to SEQ ID NO: 25

SEQ ID NO: 32 - protein, 35 aa, Amino acid
CNLTIEEFRLTYLDYYLDNIIDLFNCLANASSFSL

SEQ ID NO: 33 - protein, 35 aa, Amino acid; identical to SEQ ID NO: 32

SEQ ID NO: 34 - protein, 36 aa, Amino acid
HNLTIEEFRLAYIDNYSSKDSIDLLNCLADISKISL

SEQ ID NO: 35 - protein, 35 aa, Amino acid
CNLTIEQFRIAYLDKFSGDDTDLFNCLANVSVISL

SEQ ID NO: 36 - protein, 35 aa, Amino acid
CNLIIEKFRIAYFDKFSEDAIDSFNCLANVSTISL

SEQ ID NO: 37 - protein, 35 aa, Amino acid
CNLTIEKFRIAYFDSFSKDTTNLFNQLVNISAISL

SEQ ID NO: 38 - protein, 34 aa, Amino acid
CKVTIEEFRFTYANEFSEDITDFDCLANVSAMSL

SEQ ID NO: 39 - protein, 34 aa, Amino acid
CNVSIDEFRLTYINHFSDDIYNLNCLANISAMSF

SEQ ID NO: 40 - protein, 34 aa, Amino acid
CDVTIDEFRLTHTNDFSDDIVKFHCLANVSAMSL

(19) United States
(12) Patent Application Publication
(10) Pub. No.: US 2006/0014150 A1
(43) Pub. Date: Jan. 19, 2006

(54) Development of therapeutics for the treatment of endotoxin-meidated diseases

(75) Inventor: Walter Messier, Longmont, CO (US)

Correspondence Address: SWANSON & BRATSCHUN L.L.C., 1745 SHEA CENTER DRIVE, SUITE 330, HIGHLANDS RANCH, CO, 80129, US

(73) Assignee: Evoluntionary Genomics LLC, Aurora, CO (US)

(21) Appl. No.: 10/532,153

(22) PCT Filed: Nov. 3, 2003
(86) PCT No.: PCT/US03/36247

§ 371 Date: Apr. 20, 2005

Related U.S. Application Data
(60) Provisional application No. 60/423,113, filed on Nov. 1, 2002.

(51) Int. Cl. C12Q 1/68

(52) U.S. Cl. 435/6

(57) ABSTRACT

The subject invention comprises a method for identifying an evolutionarily meaningful nucleotide change in a primate's TLR4 polynucleotide. It further comprises methods for identifying agents that interact with the corresponding evolutionarily meaningful amino acid change so as to modulate the function of the TLR4 polypeptide, thereby attenuating activation of the NF-kB pathway. Such agents are useful in mitigating the LPS mediated response and in the treatment of sepsis, severe sepsis and septic shock.

### FIELD OF THE INVENTION

The present invention relates to methods to develop agents for treating endotoxin-mediated diseases of humans, such that the therapeutic agent interacts with the human TLR4 polypeptide extracellular domain in such a way as to attain a response resembling that of the Old World monkeys, specifically baboons and rhesus monkeys.

### BACKGROUND OF THE INVENTION

Sepsis is a serious medical condition. According to R. A. Balk and L. C. Casey (April 2000 Critical Care Clinics):
Sepsis results in 120,000 to 200,000 deaths annually in the United States
Death due to this disease (4.2 deaths/100,000) has increased 82.6% from 1979 to 1997.
It is the 12th leading cause of death overall and is the most common cause of shock encountered by internists in the U.S.
Between 300,000 to 500,000 cases of sepsis are diagnosed per year
Shock develops in about 40% of septic patients.
Despite aggressive treatment, mortality ranges from 16% in patients with sepsis to 40-60% in patients with septic shock
The annual health care cost from caring for patients with sepsis is $5-10 billion

To date, no truly effective therapy exists to counteract the effects of sepsis, although some techniques do show limited utility. An effective therapeutic approach would have tremendous social and commercial value. Described here is a method to develop to such a therapeutic.

Severe sepsis, also known as septic syndrome, refers to a chain of events leading from microbial infection to tissue injury and cardiovascular collapse. J. S. Stapczynski provides the following definitions:
“Sepsis” is the systemic host response to infection, defined as SIRS (systemic inflammatory response syndrome) in combination with a documented infection
“Severe sepsis” is defined as sepsis plus end-organ dysfunction or hypoperfusion
“Septic shock” is defined as sepsis with hypotension, despite fluid resuscitation, and evidence of inadequate tissue perfusion

Significant complications from sepsis include central nervous system dysfunction, adult respiratory distress syndrome (ARDS), liver failure, acute renal failure (ARF), and disseminated intravascular coagulation (DIC). In different studies, the reported incidence rates of these complications in SIRS and sepsis is about 19% for CNS dysfunction, 2-8% for ARDS, 12% for liver failure, 9-23% for ARF, and 8-18% for DIC (Stapczynski, J. S. 2001 eMedicine Journal 2:7).

According to N. R. Chamberlain (2001 Bacterial sepsis with shock in Infectious Diseases Lectures), sepsis involves a very complex sequence of events and much remains incompletely understood about how a patient goes from SIRS to septic shock. Patients with septic shock have a biphasic immunological response. Initially they manifest an overwhelming inflammatory response to an infection. This is probably due to the pro-inflammatory cytokines tumor necrosis factor (TNF), IL-1, IL-12, Interferon gamma (IFNγ), and IL-6). The body then regulates this response by producing anti-inflammatory cytokines (IL-10), soluble inhibitors (TNF receptors, IL-1 receptor type II, and IL-1RA, an inactive form of IL-1). The patient manifests a period of immunodepression. Persistence of this hyporesponsiveness is associated with increased risk of nosocomial infection and death.

Approximately one half of septic shock cases are caused by Gram-negative bacteria (Balk, R. A., and Casey, L. C., April 2000 Critical Care Clinics). It has long been known that sepsis can be triggered by cell-wall components of Gram-negative bacteria, termed endotoxin (Takeda, K., and Akira, S. 2001 Genes to Cells 6:733-742).

Endotoxin is also associated with the development and progression of asthma, as well as other types of airway disease (Arbour, N. C. et al., 2000 Nature Genetics 25:187-191). In asthma and airway disease, endotoxin is believed to influence pathophysiological effects of air pollution (Arbour, N. C. et al., 2000 Nature Genetics 25:187-191). The incidence of asthma and airway diseases is increasing and, like sepsis, new treatments are needed. Effective therapeutics for these endotoxin-mediated diseases represent a serious unmet medical need.

Endotoxins are composed of a lipopolysaccharide (LPS) complex, containing Lipid A and polysaccharide. The TLR4 protein has been documented (Takeda, K., and Akira, S. 2001 Genes to Cells 6:733-742) to recognize and bind LPS. This initiates a molecular cascade that triggers the innate immune system. Human TLR4 is known to be a homolog of the Drosophila protein Toll. Toll, like its human homolog, is necessary to initiate the innate immune response. Both Toll and TLR4 are known to signal through the NF-κB pathway (Medzhitov, R. et al., 1997 Nature 388:394-397).

One possible therapeutic avenue would involve inhibiting either the TLR4 gene or, more likely, the TLR4 protein, (or perhaps administration of molecules that competitively inhibit TLR4). However, this is likely to have severe and undesirable side effects. Mice strains such as C3H/HeJ and C57BL/10ScCr are unresponsive to LPS, in contrast to wild type mice, as a result of genetic defects in TLR4 (Rehli, M. et al., 2000 Journal of Biological Chemistry 275: 9773-9781). However, these strains are hypersensensitive to infection by Gram-negative bacteria (Beutler, B. 2002 Current Opinion in Hematology 9:2-10). Without a functional TLR4, and the innate immune response it triggers (which leads to an acquired immune response), these mice are unable to recognize these pathogenic invaders.

Most mammals are susceptible to septic shock. Humans, chimpanzees and bonobos are alike in extreme sensitivity to LPS, and to septic shock (Veloso, D. 1996 Immunopharmacology 33(1-3): 374-376. However, it has been well established that baboons and rhesus monkeys are resistant to septic shock, even when confronted with very high levels of LPS (Redl, H. et al., 1993 Immunobiology 187:330-345; Veloso, D. 1996 Immunopharmacology 33(1-3): 374-376). In fact, all the Old World monkeys may share this resistance to high levels of endotoxin-induced septic shock. Yet, in baboons and rhesus the innate immune response is known to be essentially the same as that of humans. Thus, baboons and rhesus have developed some mechanism for resistance to septic shock that does not interfere with innate immunity.

Because TLR4 protein is involved in septic shock, the inventors reasoned that differences in septic shock sensitivity between humans and baboons might be the result of subtle differences in the TLR4 protein. Thus, information about the specific amino acid replacements that occurred during evolution could provide unparalleled insights into the mechanism by which baboons and rhesus monkeys resist LPS-induced septic shock while maintaining functional innate immunity.

Published TLR4 sequences from human (GenBank AF177765, XM_{—}057452, U88880, and U93091), bonobo (GenBank AF179220), and baboon (GenBank AF180964) were used to design primers for polymerase chain reaction (PCR) amplification of a set of TLR4 homologs from various primates. The primate TLR4 homologs that were amplified and sequenced included rhesus monkey, gorilla, chimpanzee, gibbon, squirrel monkey, and capuchin. In addition, TLR4 was amplified and sequenced from human, bonobo, and baboon and the published sequences for these species were confirmed (Seq ID: 1 to 7). As noted in Table 1, in most cases only exons 2 and 3 were sequenced (these include the full coding region of the TLR4 gene).

TABLE 1
TLR4 Sequences

| Seq. 1 | Seq. 2 | Seq. 3 | Seq. 4 | Seq. 5 | Seq. 6 | Seq. 7 |
|---|---|---|---|---|---|---|
| Chimpanzee | Chimpanzee | Gorilla | Gibbon | Rhesus | Capuchin | Squirrel |
| (Bonnie) | (Dustin) | | | monkey | | monkey |
| Exon 3 | Exons 2 & 3 | Exons 2 & 3 | Exons 2 & 3 | Exons 2 & 3 | Exon 3 | Exons 2 & 3 |

These sequences were aligned and a series of molecular evolution analyses were then performed. Included in these analyses were Ka/Ks pairwise comparisons for each of these genes. Such pairwise comparisons calculate the differences between values of nonsynonymous nucleotide substitutions per nonsynonymous site (Ka) to synonymous substitutions per synonymous site (Ks). Ka values statistically significantly greater than the corresponding Ks values (Ka-Ks) strongly suggest the action of positive selection. Conversely, Ka values statistically significantly less than the corresponding Ks values (Ka-Ks) strongly suggest the action of negative selection, i.e., evolutionary conservation. For convenience, these pairwise comparisons are most often displayed as ratios (Ka/Ks), such that Ka/Ks>1 signifies positive selection, while Ka/Ks<1 signifies conservation.

All of these coding sequence comparisons exhibited Ka/Ks ratios less than one, some with statistical significance. This is good evidence that these are generally well-conserved, which is a commonly observed pattern. However, even well-conserved proteins can have a limited number of amino acid changes in key domains that significantly affect the function of the protein. In such cases, Ka/Ks analysis of the entire coding sequence may indicate conservation, while Ka/Ks analysis of individual domain coding regions may indicate a positively selected domain within a conserved protein. Thus, polynucleotide sequences encoding individual domains of the TLR4 protein were also subjected to Ka/Ks analysis. Two key domains are an intracellular domain responsible for signaling and an extracellular domain responsible for LPS binding.

Intracellular signaling. Ka/Ks analysis of the polynucleotide coding sequence for the TIR domain, which is the intracellular domain of TLR4 protein responsible for signaling, and which initiates the NF-κB pathway, indicates that this domain is extremely well conserved. In fact, this analysis revealed some of the lowest Ka/Ks ratios ever documented. This indicates extreme evolutionary conservation, and strongly suggests two inferences. First, this domain is a crucial one, and generally cannot tolerate amino acid replacements. Second, the signaling pathway is likely to be unchanged in all these primates. That is, regardless of differences in LPS sensitivity, the cascade initiated by the TIR domain is likely to be biochemically similar in both humans and baboons. This result thus suggests that close attention must be paid to the extracellular domain of the TLR4 protein which governs LPS recognition.

Extracellular LPS binding. LPS is thought to bind to an extracellular domain. The extracellular binding domain of TLR4 includes a number of leucine-rich repeats (LRR). These are conserved between human, bonobo, and baboon, suggesting that the basic binding mechanism is unchanged between these species. In fact, the basic LRR structure is conserved even in the Toll homolog in Drosophila. However, Ka/Ks analysis performed on the LPS binding domain for each primate TLR4 protein indicated the baboon LPS binding domain may be positively selected relative to the human or bonobo LPS binding domain, although there was only one nonsynonymous change, thus the result was not statistically significant. This is suggestive but inconclusive evidence that the difference in septic shock sensitivity between humans and baboons results from specific amino acid replacements in the LPS binding domain.

Ka/Ks analysis of the whole protein or critical domains did not provide conclusive information about the difference in sepsis susceptibility is humans versus baboons and rhesus monkeys, so we next looked at a specific amino acid of the TLR4 gene. One human TLR4 mutation (the “human null mutation”) in the extracellular ligand binding domain has been reported (Arbour, N.C. et al., 2000 Nature Genetics 25:187-191) that results in complete lack of sensitivity to LPS. The TLR4 gene from these individuals has been sequenced, and is available from GenBank (GenBank Acc. #1777766). Like baboons and rhesus, such individuals are resistant to septic shock (Arbour, N. C. et al., 2000 Nature Genetics 25:187-191). However, humans who are homozygous for this mutation have compromised immune systems and, like the C3H/HeJ and C57BL/10ScCr mice, LPS does not trigger innate immunity leaving them prone to serious Gram-negative bacterial infections.

The human null mutation is replacement of Asp299 by Gly299. Clearly, such a replacement results in substantial steric changes, leading to the loss of function observed in individuals with this mutation. Importantly, Asp299 is conserved in all mammalian TLR4s for which coding sequence data are available (except as noted below), even as phylogenetically distant from humans as mouse and rat. Such extensive conservation implies strong functional importance: this site does not generally tolerate amino acid substitutions. Importantly, however, we found that baboons and rhesus monkeys, and probably all Old World monkeys, do have an amino acid replacement at this site (Asp299 to Asn299).

| | Amino acid | Septic | Innate |
|---|---|---|---|
| | 299 | Shock | Immunity |
| Humans, most mammals, | Asp | + | + |
| Drosophila | | | |
| Human null mutation | Gly | − | − |
| Old World monkeys | Asn | − | + |
| (Rhesus, baboons, etc.) | | | |

It is clear that inhibiting TLR4 function completely is not a viable therapeutic approach because it results in too great an impairment of the immune response. Similarly, modeling a therapeutic based on the human TLR4 Gly299 mutation also would result in susceptibility to Gram-negative bacterial infections. In contrast, baboons both resist septic shock and are fully capable of recognizing and addressing Gram-negative bacterial infections. This leads to a novel approach for developing therapeutic agents for treatment of endotoxin-mediated diseases of humans.

### SUMMARY OF THE INVENTION

The subject invention comprises a method of identifying an evolutionarily meaningful nucleotide change in a first primate's polynucleotide wherein the first primate's polynucleotide may be associated with a physiological condition that is present or enhanced in the first primate relative to a second primate, comprising the steps of: (a) comparing polynucleotide sequences of the first primate with corresponding polynucleotide sequences of the second primate to identify a polynucleotide that has been overall negatively selected in the first and second primates; and (b) identifying in the first primate's overall negatively selected polynucleotide, an evolutionarily meaningful nucleotide change, whereby the nucleotide change in the first primate's negatively selected polynucleotide may be associated with the physiological condition in the first primate.

The evolutionarily meaningful nucleotide change is a nonsynonymous nucleotide change in an otherwise conserved polynucleotide that is or is believed to be associated with a physiological condition. The analysis of the polynucleotides to determine whether they are negatively selected or conserved can be carried out by any method known in the art, but preferably is accomplished by a KA/KS-type analysis as described herein.

A nucleotide change in a primate's negatively selected polynucleotide can be correlated with a physiological condition in the primate by analyzing the functional effect of the presence or absence of the identified nonsynonymous nucleotide change in a model in vivo, ex vivo or in vitro system using methods known in the art.

In one embodiment, the first primate is an Old World monkey, the second primate is Homo sapiens, and the negatively selected polynucleotide is TLR4 polynucleotide. The evolutionarily meaningful nucleotide change in the TLR4 polynucleotide results in an Asp299 in the human and an Asn299 in the Old World monkey, e.g., baboon and rhesus monkey.

The present invention also relates to methods to develop agents for treating endotoxin-mediated diseases of humans, such that the therapeutic agent interacts with the human TLR4 polypeptide extracellular domain in such a way as to attain a response resembling that of the Old World monkeys, specifically baboons and rhesus monkeys.

It has been suggested (Arbour, N. C. et al., 2000 Nature Genetics 25:187-19 1) that the region of the TLR4 receptor around residue 299 has an α-helical structure. The substitution of the glycine residue (as found in the human null mutant) for the aspartic acid residue likely disrupts the 3-D structure of this helix in a catastrophic manner. However, the asparagine residue found at position 299 in the baboon and rhesus sequences is a biochemically conservative replacement compatible with the helical structure. This evolutionarily tolerated, structurally-conservative replacement allows baboons and rhesus monkeys (and most likely, all the Old World monkeys) to modulate the interaction with LPS, such that Gram-negative bacteria still trigger the innate immune response in such a way that the known resistance of both baboon and rhesus to extremely high levels of LPS is achieved. An alignment of TLR4 protein sequences for the region of the protein that flanks this residue (from a number of mammalian species) is shown in FIG. 1.

It is believed that the Asp to Asn amino acid replacement at position 299 confers resistance to septic shock in baboons. Therefore, transgenic mice whose TLR4 encodes the Asn replacement, when compared to controls (transfected with ‘normal’ mouse TLR4), should show that the experimental transgenics exhibit an increased resistance to septic shock, i.e., they will tolerate much higher levels of LPS and/or live bacteria.

The insight of the invention described herein is that the Asp299 residue is critical to initiation of the LPS-triggered cascade that leads to endotoxin-mediated diseases, such as septic shock and asthma and other airway disorders. In one preferred embodiment, a therapeutic agent is developed that, when administered, causes human TLR4 to react to exposure to endotoxin in the way baboon or rhesus monkey TLR4 does. Accordingly, in one aspect, a method is provided whereby a peptide therapeutic agent could be isolated. Such a peptide would reduce access by LPS to the key amino acid of TLR4 determining septic shock, Asp299. If delivered during an episode of acute septic shock, the peptide should “derail” the cascade that is initiated when LPS and/or live bacteria encounters the human TLR4 protein. Such a peptide agent can be easily tested in rodent models. Successful demonstration of protection of such models from septic shock would pave the way to similar human trials of such peptide agent. Because such a peptide would only be administered during acute episodes of septic shock, possible problems stemming from repeated administration and subsequent sensitization would be minimized. Those skilled in the art can easily determine the optimal length and amino acid composition of such therapeutic peptide, which can be further refined by testing in rodent models, using methods known in the art.

In another embodiment, a therapeutic peptide could be designed that had the same sequence as the region surrounding Asp299. Such a therapeutic could be useful as a decoy to bind to LPS and reduce the amount of LPS available to bind to the TLR4 protein.

In another embodiment, an antibody or portion of an antibody could be isolated or designed that could attenuate access by endotoxin to Asp299 such that the endotoxin-mediated cascade is reduced. In a preferred embodiment, the antibody or fragment thereof is directed to an epitope that includes the Asp299 residue; the epitope preferably is an amino acid segment of 10 or less residues containing the Asp299 residue.

In another embodiment, a small molecule is identified that will reduce access to the critical Asp299 residue, or modulate the interaction of endotoxin with the Asp299 residue such that the cascade leading to endotoxin-mediated disease is modulated.

Another embodiment is to use the method disclosed herein to develop a therapeutic agent to treat human asthma. Small molecules could be designed or identified by screening libraries of small molecules that interact with Asp299 of the TLR4 polypeptide or the region containing the Asp299 polypeptide. Such therapeutic agents could be used to ameliorate the severity of asthmatic episodes. It is also likely that some therapeutic agents identified using the methods of this invention could be administered on a regular basis to reduce the effects of chronic airway diseases.

Using the teachings provided herein, persons skilled in the art will recognize that therapeutics can be developed for other diseases involving LPS-TLR4 protein interactions.

All references cited herein are each incorporated herein by reference in their entirety.

### BRIEF DESCRIPTION OF THE DRAWINGS

FIG. 1 is an alignment of TLR4 protein sequences for the region of the protein that flanks the Asp299 residue from a number of mammalian species. Amino acid residues are shown in the single letter IUPAC code. Residues that are identical in all species examined are shown in bold. Dashes have been introduced (where insertions or deletions have occurred) to maximize the alignment. The critical residue (human Asp299, baboon Asn299) is shown in lower case. Note that this Asp residue is conserved in all mammal species examined, with the exception of the biochemically-conservative Asn replacement in the Old World monkeys baboon and rhesus (and, importantly, the non-functional human null mutant).

FIG. 2 is the nucleotide sequence for a first chimpanzee's (“Bonnie”) TLR4 exon 3.

FIG. 3 is the nucleotide sequence for a second chimpanzee's (“Dustin”) TLR4 exons 2 and 3.

FIG. 4 is the nucleotide sequence for gorilla TLR4 exons 2 and 3.

FIG. 5 is the nucleotide sequence for gibbon TLR4 exons 2 and 3.

FIG. 6 is the nucleotide sequence for rhesus monkey TLR4 exons 2 and 3.

FIG. 7 is the nucleotide sequence for capuchin TLR4 exon 3.

FIG. 8 is the nucleotide sequence for squirrel monkey TLR4 exon 3.

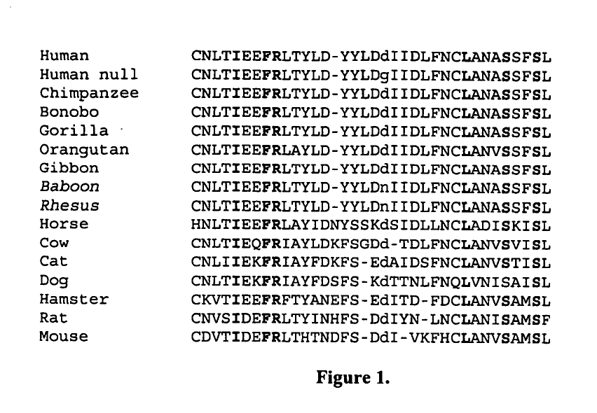

### DETAILED DESCRIPTION OF THE INVENTION

The subject invention relates to a method of identifying a nucleotide change in a TLR4 polynucleotide sequence of an Old World monkey wherein such change may be associated with reduced sensitivity to Gram-negative bacterial infection. This method involves the comparison of the TLR4 polynucleotide sequence from the Old World monkey with corresponding TLR4 polynucleotide sequence of a human to identify a polynucleotide change in said Old World monkey's TLR4 sequence that is evolutionarily meaningful. The evolutionarily meaningful change may then be associated with reduced sensitivity to Gram-negative bacterial infection. In particular, the evolutionarily meaningful change is from Asp299 in the human to Asn299 in the rhesus monkey or baboon.

The subject invention also includes a method of identifying a therapeutic agent that reduces sensitivity to Gram-negative bacterial infection. This method comprises (a) contacting candidate agents with human TLR4 polypeptide; and (b) identifying a therapeutic agent that interacts with the TLR4 polypeptide to substantially reduce sensitivity to Gram-negative bacterial infection. The agent interaction with TLR4 polypeptide preferably occurs at Asp299.

The therapeutic agent identified according to the subject invention can be used in the treatment of sepsis, severe sepsis, septic shock, asthma or other respiratory ailments, in humans or non-human primates.

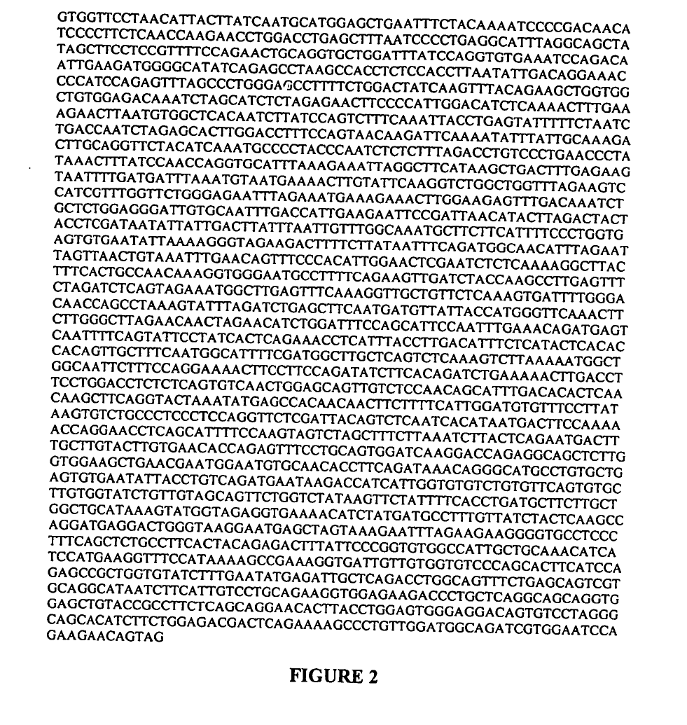

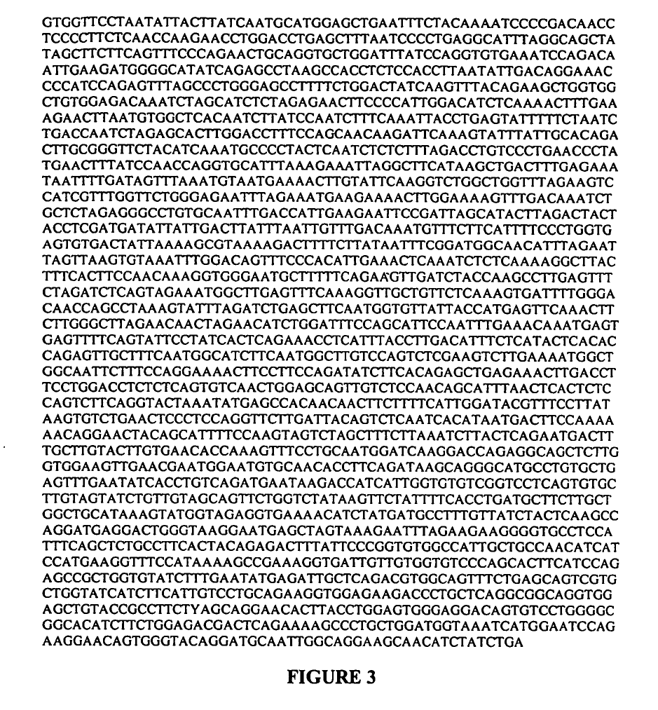

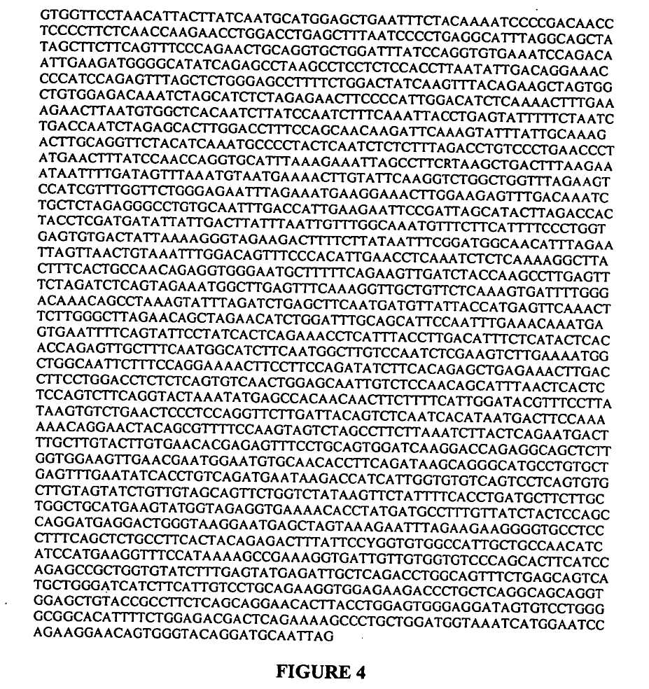

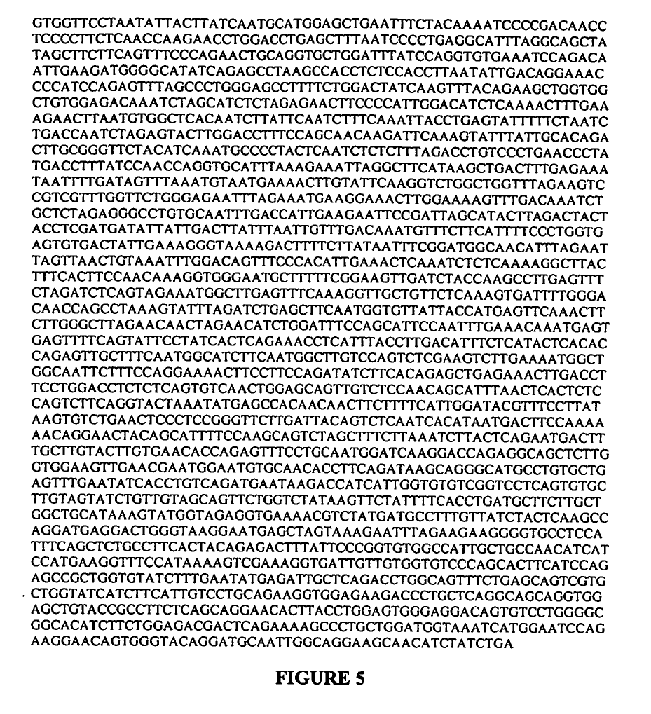

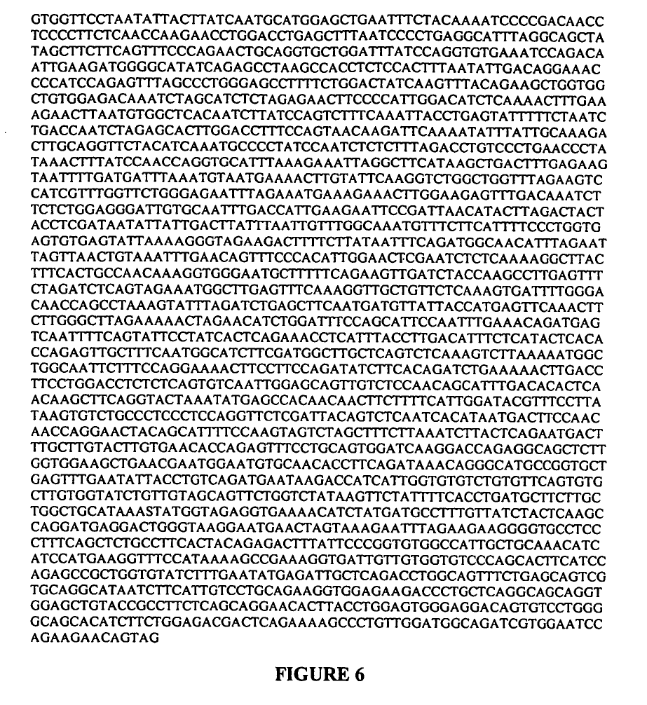

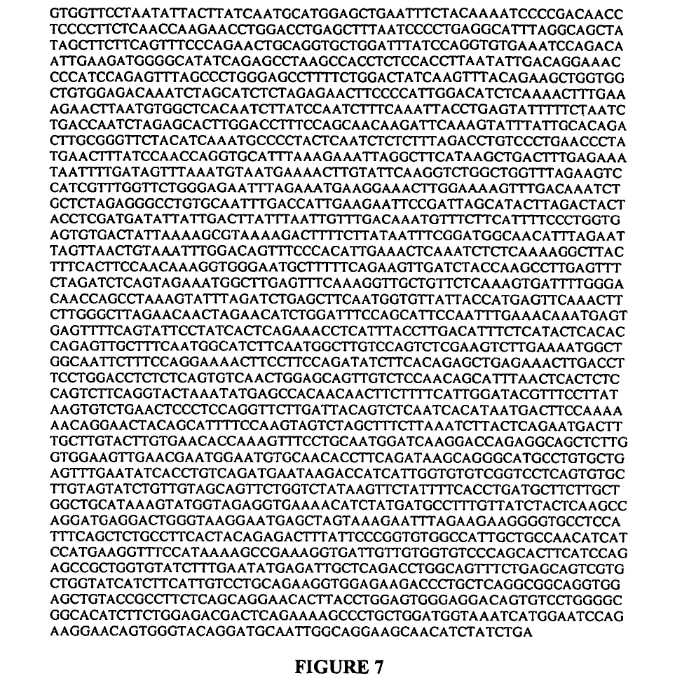

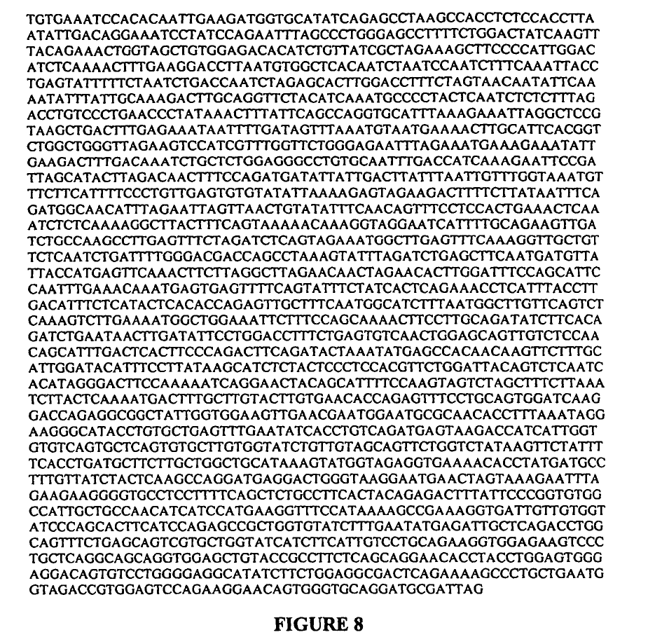

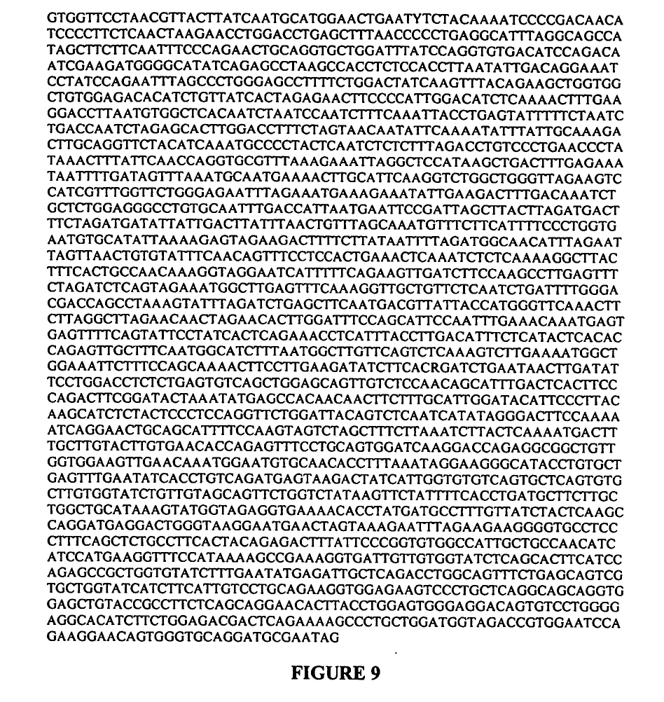

The subject invention also concerns a number of novel TLR4 polynucleotide sequences as provided in FIGS. 2-9, and their deduced TLR4 polypeptide sequences. These sequences are chimpanzee, gorilla, gibbon, rhesus monkey, capuchin, squirrel monkey and baboon. They are useful in the identification of evolutionarily meaningful nucleotide changes in other primate TLR4 polynucleotides. Their polynucleotide or polypeptide sequences may also be useful in the design of candidate therapeutic agents according to the subject invention.

The practice of the present invention employs, unless otherwise indicated, conventional techniques of molecular biology, genetics, and assay development, which are within the skill of the art. Such techniques are explained fully in the literature, such as: “Molecular Cloning: A Laboratory Manual”, second edition (Sambrook et al., 1989); “Oligonucleotide Synthesis” (M. J. Gait, ed., 1984); “Current Protocols in Molecular Biology” (F. M. Ausubel et al., eds., 1987); “PCR: The Polymerase Chain Reaction”, (Mullis et al., eds., 1994)

### DEFINITIONS

As used herein, a “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified polynucleotides such as methylated and/or capped polynucleotides. The terms “polynucleotide” and “nucleotide sequence” are used interchangeably.

As used herein, a “gene” refers to a polynucleotide or portion of a polynucleotide comprising a sequence that encodes a protein. It is well understood in the art that a gene also comprises non-coding sequences, such as 5′ and 3′ flanking sequences (such as promoters, enhancers, repressors, and other regulatory sequences) as well as introns.

The terms “polypeptide,” “peptide,” and “protein” are used interchangeably herein to refer to polymers of amino acids of any length. These terms also include proteins that are post-translationally modified through reactions that include glycosylation, acetylation and phosphorylation.

The term “K_{A}/K_{S}-type methods” means methods that evaluate differences, frequently (but not always) shown as a ratio, between the number of nonsynonymous substitutions and synonymous substitutions in homologous genes (including the more rigorous methods that determine non-synonymous and synonymous sites). These methods are designated using several systems of nomenclature, including but not limited to K_{A}/K_{S}, d_{N}/d_{S}, D_{N}/D_{S}.

The term “evolutionarily meaningful change” refers to one or more nonsynonymous nucleotide change(s) or corresponding amino acid change(s) between two species that occurs in an otherwise conserved polynucleotide or polypeptide, that may be attributed to a positive selective pressure, and which is or is believed to be associated with a physiological condition. A conserved polynucleotide can be identified by methods known in the art including a K_{A}/K_{S}-type analytical method. Typically, a K_{A}/K_{S} ratio less than about 1.0, more preferably less than about 0.75, and most preferably less than about 0.5 indicates the action of negative selection. The presence of a nonsynonymous nucleotide change in such a conserved polynucleotide (i.e., containing no other nucleotide changes or only synonymous nucleotide changes) is considered to be an evolutionarily meaningful change. The phrase “associated with a physiological condition” means that the nonsynonymous nucleotide change has been observed in individuals to result in the physiological condition at issue, has been shown to be involved in a molecular mechanism related to the physiological condition, and/or occurs in a location of the gene that is relevant to a protein function that is essential to the occurrence of the physiological condition. For example, as discussed herein, a nonsynonymous nucleotide change in the baboon or rhesus monkey gene is believed to be associated with the physiological condition of enhanced resistance to the endotoxin-mediated response.

The term “positive evolutionarily meaningful change” means an evolutionarily meaningful change in a particular species that results in an adaptive change that is positive as compared to other related species. An example of a positive evolutionarily meaningful change is a change that has resulted in reduced sensitivity to the LPS mediated response.

The term “resistant” means that an organism exhibits an ability to avoid, or diminish the extent of, a disease condition and/or development of the disease, preferably when compared to non-resistant organisms.

The term “susceptibility” means that an organism fails to avoid, or diminish the extent of, a disease condition and/or development of the disease condition, preferably when compared to an organism that is known to be resistant.

It is understood that resistance and susceptibility vary from individual to individual, and that, for purposes of this invention, these terms also apply to a group of individuals within a species, and comparisons of resistance and susceptibility generally refer to overall, average differences between species, although intra-specific comparisons may be used.

The term “nucleotide change” refers to nucleotide substitution, deletion, and/or insertion, as is well understood in the art.

The term “agent”, as used herein, means a biological or chemical compound such as a simple or complex organic or inorganic molecule, a peptide, a protein or an oligonucleotide that modulates the function of a polypeptide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. The term “agent” can include or exclude antibodies or fragments thereof. Compounds can be tested singly or in combination with one another.

The term “therapeutic agent” as used herein means an agent as described above used to treat a disease or condition.

The term “to modulate function” of a polypeptide means that the function of the polypeptide is altered in the presence of an agent compared to the absence of the agent. Modulation may occur on any level that affects function. Modulation of a polypeptide function may be direct or indirect, and measured directly or indirectly.

A “function of a polypeptide” includes, but is not limited to, conformation, folding (or other physical characteristics), binding to other moieties (such as ligands), activity (or other functional characteristics), and/or other aspects of protein structure or functions. For example, an agent that acts on a polypeptide and affects its conformation, folding (or other physical characteristics), binding to other moieties (such as ligands), activity (or other functional characteristics), and/or other aspects of protein structure or functions is considered to have modulated polypeptide function. The ways that an effective agent can act to modulate the function of a polypeptide include, but are not limited to 1) changing the conformation, folding or other physical characteristics; 2) changing the binding strength to its natural ligand or changing the specificity of binding to ligands; and 3) altering the activity of the polypeptide.

The term “to modulate the endotoxin or LPS mediated response” means that the function of the TLR4 polypeptide is altered in the presence of an agent compared to the absence of the agent. The modulation reduces the clinical symptoms of sepsis, severe sepsis, or septic shock, including central nervous system dysfunction, adult respiratory distress syndrome, liver failure, acute renal failure, disseminated intravascular coagulation, and the like. Preferably, these symptoms are reduced in increasing preference by at least about 10%, 20%, 30%, 40%, 50%, 60%, 70%, 80%, 90% and 100%, as measured by standard clinical indicators or assays for those symptoms as is known in the art. Modulation can be detected in other ways as well, including reduced affinity or altered kinetics of LPS binding to the TLR4 extracellular domain, or reduced signaling to a downstream effector of TLR4 in the LPS mediated response. Preferably, the agent modulation results in the attenuation of the LPS mediated response to the degree that the human TLR4 interacts with the LPS in the same manner as the Old World monkey TLR4.

Further, “modulation of endotoxin or LPS mediated response” means significant reduction or attenuation of the response whereby the clinical symptoms of sepsis, severe sepsis or septic shock are reduced as indicated above. It does not refer to abrogation or elimination of the response; i.e., innate immunity remains intact. The therapeutic agent of the subject invention is one which interacts directly or indirectly with the Asp299 residue such that the activation of the NF-kB pathway and the clinical symptoms of sepsis, severe sepsis and septic shock are attenuated.

The term “target site” means a location in a polypeptide which can be a single amino acid and/or is a part of, a structural and/or functional motif, e.g., a binding site, a dimerization domain, or a catalytic active site. Target sites may be useful for direct or indirect interaction with an agent, such as a therapeutic agent.

The term “positively selected” means an evolutionarily significant change in a particular organism, species, subspecies, variety, cultivar or strain that results in an adaptive change that is positive as compared to other related organisms. An example of a positive evolutionarily significant change is a change that has resulted in enhanced yield in crop plants. As stated above, positive selection is indicated by a K_{A}/K_{S} ratio greater than 1.0. With increasing preference, the K_{A}/K_{S} value is greater than 1.25, 1.5 and 2.0.

“TLR4” as used herein refers to the polynucleotide encoding the Toll like receptor 4 polypeptide.

“TLR4” as used herein refers to the polypeptide encoded by TLR4 polynucleotide.

“LPS” as used herein refers to lipopolysaccharide and is used interchangeably with the word “endotoxin”.

### GENERAL METHODS OF THE INVENTION

The general method of the invention is as follows. Briefly, the TLR4 polynucleotide sequences are obtained from a human source and a number of non-human primate sources. They are compared to one another to determine whether the TLR4 polynucleotide is conserved or negatively selected. Then, having determined that the polynucleotide is conserved, the TLR4 polynucleotide sequences from the human and other primates are analyzed to identify any nonsynonymous or evolutionarily meaningful nucleic acid differences. The TLR4 sequences from each species are then characterized in terms of whether they do or do not correlate with decreased sensitivity to Gram-negative bacterial infection for that species, thereby indicating those evolutionarily meaningful changes that could be or are associated with the decreased sensitivity to LPS. This method resulted in the identification of Asn299 as the critical amino acid found in baboons and rhesus monkeys that confers attenuated sensitivity to LPS in those species as compared to humans which have Asp299.

U.S. Ser. No. 10/100,422, filed Mar. 18, 2002, and incorporated herein in its entirety by reference, describes in detail a number of methods useful in sequencing homologous polynucleotide sequences from primates, and methods for identification of evolutionarily meaningful changes in polynucleotides that can be correlated with a particular physiological condition in humans or in non-human primates.

Determination of evolutionarily meaningful changes first requires a determination as to whether the polynucleotide at issue is negatively selected. Any of several different molecular evolution analyses or K_{A}/K_{S}-type methods can be employed to determine whether the human gene sequences and the non-human primate polynucleotide are conserved or negatively selected. Kreitman and Akashi (1995) Annu. Rev. Ecol. Syst 26:403-422; Li, Molecular Evolution, Sinauer Associates, Sunderland, Mass., 1997. For example, negative selection on proteins (i.e., molecular-level conservation) can be detected in protein-coding genes by pairwise comparisons of the ratios of nonsynonymous nucleotide substitutions per nonsynonymous site (K_{A}) to synonymous substitutions per synonymous site (K_{S}) (Li et al., 1985; Li, 1993). Any comparison of K_{A} and K_{S} may be used, although it is particularly convenient and most effective to compare these two variables as a ratio. Negatively selected polynucleotides are identified by having a K_{A}/K_{S} ratio of less than 1.0, preferably less than 0.75 and more preferably less than 0.5. Preferably, the K_{A}/K_{S} analysis by Li et al. is used to carry out the present invention, although other analysis programs that can detect negatively selected genes between species can also be used. Li et al. (1985) Mol. Biol Evol. 2:150-174; Li (1993); see also J. Mol. Evol. 36:96-99; Messier and Stewart (1997) Nature 385:151-154; Nei (1987) Molecular Evolutionary Genetics (New York, Columbia University Press). The K_{A}/K_{S} method, which comprises a comparison of the rate of non-synonymous substitutions per non-synonymous site with the rate of synonymous substitutions per synonymous site between homologous protein-coding region of genes in terms of a ratio, can be used to identify conserved polynucleotides. A synonymous (“silent”) substitution is one that, owing to the degeneracy of the genetic code, makes no change to the amino acid sequence encoded; a nonsynonymous substitution results in an amino acid replacement. The extent of each type of change can be estimated as K_{A} and K_{S}, respectively, the numbers of synonymous substitutions per synonymous site and non-synonymous substitutions per non-synonymous site. Calculations of K_{A}/K_{S} may be performed manually or by using software. An example of a suitable program is MEGA (Molecular Genetics Institute, Pennsylvania State University).

For the purpose of estimating K_{A} and K_{S}, either complete or partial human and primate protein-coding sequences are used to calculate total numbers of synonymous and non-synonymous substitutions, as well as non-synonymous and synonymous sites. The length of the polynucleotide sequence analyzed can be any appropriate length, but is preferably at least 60 nucleotides in length. Preferably, the entire coding sequence is compared, in order to determine overall conservation. Publicly available computer programs, such as Li93 (Li (1993) J. Mol. Evol. 36:96-99) or INA, can be used to calculate the K_{A} and K_{S} values for all pairwise comparisons.

As indicated above, conservation is indicated by the K_{A}/K_{S} ratio being less than about 1.0, more preferably less than about 0.75, and most preferably less than about 0.5. Preferably, statistical analysis is performed on all decreased K_{A}/K_{S} ratios, including, but not limited to, standard methods such as Student's t-test and likelihood ratio tests described by Yang (1998) Mol. Biol Evol. 37:441-456.

All methods for calculating K_{A}/K_{S} ratios are based on a pairwise comparison of the number of nonsynonymous substitutions per nonsynonymous site to the number of synonymous substitutions per synonymous site for the protein-coding regions of homologous genes from related species. Each method implements different corrections for estimating “multiple hits” (i.e., more than one nucleotide substitution at the same site). Each method also uses different models for how DNA sequences change over evolutionary time. Thus, preferably, a combination of results from different algorithms is used to increase the level of sensitivity for detection of negatively-selected genes and confidence in the result.

As discussed above, the foregoing methods resulted in the identification of an evolutionarily meaningful nucleotide change in the conserved TLR4 polynucleotide. The Asn299 of baboons and rhesus monkeys was found to attenuate LPS sensitivity in those species relative to humans which have Asp299. This information can be useful in the development of agents that interact with human TLR4 Asp299 in such a manner so as to attenuate activation of the NF-KB pathway by LPS, therefore aiding in the treatment of sepsis, severe sepsis and septic shock.

An agent is designed or identified that will interact with human TLR4 protein in such a way that the agent modulates the human endotoxin mediated response. Preferably, the agent causes the human TLR4 to interact with LPS in a manner that is similar to that of baboon or rhesus TLR4 interaction with LPS. Such agents can be peptide, protein, organic molecules, or aptamers, or whatever agent can have the specific effect.

Generally, techniques of combinatorial chemistry can be used to generate numerous permutations of agent candidates to be screened for effectiveness in reducing access of LPS to Asp299 of TLR4. Those of skill in the art can devise and/or obtain suitable agents for testing. In general, screening can be performed by adding an agent to a sample of isolated TLR4 or appropriate cells expressing TLR4 and monitoring effect, that is, modulation of the cascade known to lead to endotoxin-mediated disease. The experiments preferably include a control sample which does not receive the candidate agent. Differences between treated and untreated cells indicate effects attributable to the candidate agent. Optimally, a greater effect is seen in the presence of the candidate agent than in the absence of the candidate agent. Hoshino, K., Takeuchi, O., Kawai, T., et al. (1999). J. Immunol. 162, 3749-3752. describe a typical assay for measuring the NF-kB pathway that is known to lead to endotoxin-mediated disease.

The screening methods for agents that interact with TLR4 polypeptide can be carried out in vitro, ex vivo or in vivo using TLR4 protein or polypeptide or extracellular fragment thereof, or NF-KB pathway models known in the art.

In an example for an assay for an agent that binds to TLR4 polypeptide, an affinity column is prepared with purified human TLR4 or a synthetically prepared peptide of a small region of TLR4 containing Asp299. The affinity column is then used to screen a library of compounds (libraries of compounds include, but are not limited to, peptides, aptamers, small molecules, etc.) which have been appropriately labeled. Suitable labels include, but are not limited to fluorochromes, radioisotopes, enzymes and chemiluminescent compounds. The unbound and bound compounds can be separated by washes using various conditions known to those skilled in the art. In addition to affinity columns, there are other techniques, such as measuring the fluorescence anisotropy of a protein which will change upon binding another molecule. For example, a BIAcore assay using a sensor chip (supplied by Pharmacia Biosensor, Stitt et al. (1995) Cell 80:661-670.) that is coupled to TLR4 or a peptide of TLR4 containing Asp299 may be performed to determine the binding activity of different agents.

It is also understood that the in vitro screening methods of this invention include structural, or rational, drug design, in which the amino acid sequence surrounding Asp299, three-dimensional atomic structure or other property (or properties) of the amino acid sequence surrounding Asp299 provides a basis for designing an agent which is expected to bind to TLR4 in such a way as to reduce access by endotoxin to Asp299.

The screening methods described above represent primary screens, designed to detect any agent that may bind to TLR4 and/or exhibit activity that modulates the function of TLR4. The skilled artisan will recognize that secondary tests will likely be necessary in order to evaluate an agent further. For example, a secondary screen may comprise testing the agent(s) in a mouse model or other animal models for effect in reducing the endotoxin-mediated cascade leading to disease.

The invention also includes agents identified by the screening methods described herein.

Peptide Agents

A peptide agent can be isolated by screening a library of randomly synthesized peptides for peptides that bind to residue 299, using methods known in the art, for example, as described in Dower, William J; Cwirla, Steven E; Barrett, Ronald W, “Peptide library and screening systems.” Biotechnol Advances 1996 14(4):490. Peptides found to bind to TLR4 in such a way that the endotoxin does not interact directly with Asp299 are selected. In one embodiment, the peptide library contains randomly synthesized peptides of at least 20 amino acids in length, preferably less than 50 amino acids in length. In another embodiment, the peptide library contains randomly synthesized peptides of between 15 and 20 amino acids in length. In a third embodiment, the peptide library contains peptides between 10 and 14 amino acids in length. In a fourth embodiment, the peptide library contains peptides between 1 and 9 amino acids in length. Peptides found to bind TLR4 at or near residue 299 are then subjected to secondary screens in vitro for effect on the binding of LPS to TLR4 and prevention or attenuation of TLR4 signaling.

Aptamers

Similar to that described above, the subject invention includes methods to identify an aptamer agent by screening a library of randomly synthesized single-stranded nucleotides, using methods known in the art, for example, as described in Bell, C.; Lynam, E.; Landfair, D. J.; Janjic, N.; and Wiles, M. E., “Oligonucleotides NX1838 inhibits VEGF165-mediated cellular responses in vitro”, In Vitro Cell Dev. Biol. Anim. 1999 Oct:35(9):533-42.

Small Molecule Agents

One method for developing such a small molecule agent would be to use 3-dimensional modeling of the secondary and tertiary structure of the region of TLR4 that surrounds the critical Asp299 residue. Potential small molecules can be ‘docked’ in silico, in order to identify a close fit that will inhibit access to the aspartic acid residue or have the desired structural affect. Likely candidates can then be tested in vivo in rodent models. Small molecule therapeutics could also be identified from libraries of small molecules through the use of assays that screen for compounds that bind to the region of TLR4 containing Asp299. In a similar fashion, it may be possible to design or screen for a small molecule that does not completely block access to the Asp299 residue, but rather modulates the kinetics of LPS binding in this region in such a way that it more closely resembles the kinetics of LPS binding to baboon TLR4 protein.

Therapeutic Compositions that Comprise Agents

As described herein, agents can be screened for their capacity to modulate the LPS mediated NF-kB pathway.

Various delivery systems are known in the art that can be used to administer agents identified according to the subject methods. Such delivery systems include aqueous solutions, encapsulation in liposomes, microparticles or microcapsules or conjugation to a moiety that facilitates intracellular admission.

Therapeutic compositions comprising agents may be administered parenterally by injection, although other effective administration forms, such as intra-articular injection, inhalant mists, orally-active formulations, transdermal iontophoresis or suppositories are also envisioned. The carrier may contain other pharmacologically-acceptable excipients for modifying or maintaining the pH, osmolarity, viscosity, clarify, color, sterility, stability, rate of dissolution, or odor of the formulation. The carrier may also contain other pharmacologically-acceptable excipients for modifying or maintaining the stability, rate of dissolution, release or absorption of the agent. Such excipients are those substances usually and customarily employed to formulate dosages for parenteral administration in either unit dose or multi-dose form.

Once the therapeutic composition has been formulated, it may be stored in sterile vials as a solution, suspension, gel, emulsion, solid, or dehydrated or lyophilized powder. Such formulations may be stored either in a ready to use form or requiring reconstitution immediately prior to administration. The manner of administering formulations containing agents for systemic delivery may be via subcutaneous, intramuscular, intravenous, intranasal or vaginal or rectal suppository.

The amount of agent which will be effective in the treatment of a particular disorder or condition will depend on the nature of the disorder or condition, which can be determined by standard clinical techniques. In addition, in vitro or in vivo assays may optionally be employed to help identify optimal dosage ranges. The precise dose to be employed in the formulation will also depend on the route of administration, and the seriousness or advancement of the disease or condition, and should be decided according to the practitioner and each patient's circumstances. Effective doses may be extrapolated from dose-response curves derived from in vitro or animal model test systems. For example, an effective amount of an agent identified according to the subject methods is readily determined by administering graded doses of the agent and observing the desired effect.

The following examples are provided to further assist those of ordinary skill in the art. Such examples are intended to be illustrative and therefore should not be regarded as limiting the invention. A number of exemplary modifications and variations are described in this application and others will become apparent to those of skill in this art. Such variations are considered to fall within the scope of the invention as described and claimed herein.

### Example 1

### PCR amplification and DNA Sequencing of Primate TLR4 Sequences

Published TLR4 sequences from human (GenBank AF177765, XM_{—}057452, U88880, and U93091), bonobo (GenBank AF179220), and baboon (GenBank AF180964) were used to design primers (by methods well-known to those skilled in the art) for polymerase chain reaction (PCR) amplification of a set of TLR4 homologs from various primates. The primate TLR4 homologs that were PCR amplified and DNA sequenced (by methods well-known to those skilled in the art) included rhesus monkey, gorilla, chimpanzee, gibbon, squirrel monkey, and capuchin. In addition, TLR4 was amplified and sequenced from human, bonobo, and baboon and the published sequences for these species were confirmed (Seq ID: I to 7). Because exons 2 and 3 contain the full coding region of the TLR4 gene, in most cases only exons 2 and 3 were sequenced. These sequences were aligned by methods well-known to those skilled in the art.

### Example 2

### Ka/Ks Analysis

Ka/Ks pairwise comparisons were completed for each of these genes. Such pairwise comparisons calculate the differences between values of nonsynonymous nucleotide substitutions per nonsynonymous site (Ka) to synonymous substitutions per synonymous site (Ks). Ka values statistically significantly greater than the corresponding Ks values (Ka-Ks) strongly suggest the action of positive selection. Conversely, Ka values statistically significantly less than the corresponding Ks values (Ka-Ks) strongly suggest the action of negative selection, i.e., evolutionary conservation. For convenience, these pairwise comparisons are most often displayed as ratios (Ka/Ks), such that Ka/Ks>1 signifies positive selection, while Ka/Ks<1 signifies conservation.

All of these whole protein comparisons exhibited Ka/Ks ratios less than one, some with statistical significance. This is good evidence that these are generally well-conserved proteins, which is a commonly observed pattern. However, even well-conserved proteins can have amino acid changes in key domains that significantly affect the function of the protein. In such cases, Ka/Ks analysis of the entire coding sequence may indicate conservation, while Ka/Ks analysis of individual domain coding regions may indicate a positively selected domain within a conserved protein. Thus, polynucleotides encoding individual domains of the TLR4 protein were also subjected to analysis. Two key domains are an intracellular domain responsible for signaling and an extracellular domain responsible for LPS binding. Ka/Ks analysis was performed for the TIR domain, which is the intracellular domain of TLR4 protein responsible for signaling, and which initiates the NF-κB pathway. This analysis indicated that this domain is extremely well conserved. In fact, this analysis revealed some of the lowest Ka/Ks ratios ever documented. Ka/Ks analysis was then performed for the extracellular signaling domain of TLR. Here the result was inconclusive, in that although evidence was seen for possible positive selection on the extracellular LPS-binding domain of baboon TLR4 (relative to human TLR4), no statistical support exists for this. As a result, further analysis was performed (see Example 3).

### Example 3

### Further Molecular Evolutionary Analysis

Further analysis included a search for individual amino acid replacements that are either shared by, or are unique to, the human and baboon TLR4 sequences. One human TLR4 mutation in the extracellular ligand binding domain has been reported (Arbour, N.C. et aL, 2000 Nature Genetics 25:187-191) that results in complete lack of sensitivity to LPS. Like baboons and rhesus, such individuals are resistant to septic shock. However, humans who are homozygous for this mutation have compromised immune systems and LPS does not trigger innate immunity leaving them prone to serious Gram-negative bacterial infections. The human null mutation is replacement of Asp299 by Gly299. Importantly, Asp299 is conserved in all mammalian TLR4s for which sequence is available, even as phylogenetically distant as mouse and rat, with the exception of baboon and rhesus, which have a biochemically conservative replacement amino acid replacement at this site (Asp299 to Asn299). The substitution of the glycine residue (as found in the human null mutant) for the aspartic acid residue likely disrupts the 3-D structure of this helix in a catastrophic manner. However, because the asparagine residue found at position 299 in the baboon and rhesus sequences is a biochemically conservative replacement, it is likely compatible with the helical structure. This evolutionarily tolerated, structurally-conservative replacement thus probably allows baboons and rhesus monkeys (and most perhaps, all the Old World monkeys) to modulate the interaction with LPS, such that Gram-negative bacteria still trigger the innate immune response in such a way that the known resistance of both baboon and rhesus to extremely high levels of LPS is achieved.

### Example 4

### Design of a Peptide Therapeutic Agent

A library of random peptides 20 amino acids long is synthesized and screened for peptide agents that bind to TLR4. A secondary screen then assesses peptides found in this primary screen for ability to reduce access by LPS to Asp299 and measurably reducing the LPS-mediated cascade leading to septic shock. The optimal length and amino acid composition of the therapeutic peptide agent can be refined by testing in rodent models, as would be known to one skilled in the art.

### Example 5

### Design and Synthesis of a Decoy Peptide Agent

A peptide is designed that has the same sequence as the region of human TLR4 from amino acid 289 through 309. This peptide is synthesized synthetically and formulated for delivery as a therapeutic. Such a peptide therapeutic would be useful as a decoy to bind to LPS and reduce the amount of LPS available to bind to the TLR4 protein.

### Example 6

### Design and Synthesis of a Small Molecule Therapeutic Agent

The secondary and tertiary structure of the region of TLR4 protein from amino acid 289 through 309 is modeled. Small molecule core structures are screened in silico for the ability to ‘dock’ in this region in order to identify a close fit that will inhibit access by endotoxin to the Asp299 residue either by directly or indirectly reducing access to Asp299. Likely candidates can then be tested in vivo in rodent models.

### Example 7

### Screening for a Small Molecule Therapeutic

Libraries of small molecules are purchased from one of several vendors. An affinity column is prepared with purified human TLR4 or a synthetically prepared peptide of a small region of TLR4 containing Asp299. The affinity column is then used to screen a library of compounds which have been appropriately labeled. Suitable labels include, but are not limited to fluorochromes, radioisotopes, enzymes and chemiluminescent compounds. The unbound and bound compounds can be separated by washes using various conditions known to those skilled in the art. In addition to affinity columns, there are other techniques, such as measuring the fluorescence anisotropy of a protein which will change upon binding another molecule. For example, a BIAcore assay using a sensor chip (supplied by Pharmacia Biosensor, Stitt et al. (1995) Cell 80:661-670.) that is coupled to TLR4 or a peptide of TLR4 containing Asp299 may be performed to determine the binding activity of different agents.

A secondary screen is then employed to identify small molecules that reduce access by endotoxin to the Asp299 residue, or modulate the kinetics of endotoxin binding in the region containing Asp299 in such a way that it more closely resembles the kinetics of endotoxin binding to baboon TLR4 protein. For example, mammals that are susceptible to LPS-mediated response (i.e., those with Asp299) could be administered an appropriate dose of the candidate agent to determine if it attenuates the sepsis symptoms normally associated with exposure to LPS.

### Example 8

### Screening Antibody Candidates

Antibodies (or modified antibodies or antibody fragments) are isolated/designed that bind to the extracellular region of human TLR4 such that access by bacterial LPS is diminished to Asp299. Such antibodies are directed to an epitope comprising Asp299. Preferably the epitope is 10 or less residues in length. Creation or isolation of such antibodies is understood by those skilled in the art. Uehori et al. (2003) Infect. Immun. 71 (8):4238, describe antibodies to TLR4 which inhibited bacterial cell wall skeleton mediated NF-kB activation by 80%. Also see akashi et al. (2000) J. Immunol. 164:3471-75.

1. A method of identifying a nucleotide change in a TLR4 polynucleotide sequence of an Old World monkey wherein said change may be associated with reduced sensitivity to Gram-negative bacterial infection, comprising the step of:
comparing the TLR4 polynucleotide sequence of the Old World monkey with corresponding TLR4 polynucleotide sequence of a human to identify a polynucleotide change in said Old World monkey's TLR4 sequence that is evolutionarily meaningful, whereby said evolutionarily meaningful change may be associated with reduced sensitivity to Gram-negative bacterial infection.

2. The method of claim 1 wherein the Old World monkey is selected from the group consisting of rhesus monkey and baboon.

3. The method of claim 2 wherein the evolutionarily meaningful change is from Asp299 in the human to Asn299 in the rhesus monkey or baboon.

4. The method of claim 3, wherein the evolutionarily meaningful change is associated with the reduced sensitivity to Gram-negative bacterial infection by the step comprising:
analyzing the functional effect of the Asp299Asn change in a model system.

5. The method of claim 4, wherein said model system is in vivo, ex vivo or in vitro.

6. A method of identifying a therapeutic agent that reduces sensitivity to Gram-negative bacterial infection, comprising:
(a) contacting candidate agents with human TLR4 polypeptide; and
(b) identifying a therapeutic agent that interacts with the TLR4 polypeptide to substantially reduce sensitivity to Gram-negative bacterial infection.

7. The method of claim 6 wherein said interaction with TLR4 polypeptide occurs at Asp299.

8. The method of claim 6, wherein said substantial reduction in sensitivity to Gram-negative bacterial infection is determined by an indicator selected from the group consisting of:
(a) elimination or substantial reduction in host systemic inflammatory response to LPS in a human, non-human primate, or suitable animal model; and
(b) elimination or reduced severity of central nervous system dysfunction, adult respiratory distress syndrome, liver failure, acute renal failure, and/or disseminated intravascular coagulation in a human, non-human primate, or suitable animal model.

9. A method for treating sepsis, severe sepsis or septic shock in a primate, comprising:
administering to a primate in need thereof an effective dose of a therapeutic agent identified according to the method of claim 6.

10. A method for treating asthma in a primate, comprising:
administering to a primate in need thereof an effective dose of a therapeutic agent identified according to the method of claim 6.

11. A therapeutic agent identified according to the method of claim 6.

12. A composition comprising a polynucleotide selected from the group consisting of chimpanzee TLR4 polynucleotide (SEQ. ID. NO. 1), gorilla TLR4 polynucleotide (SEQ. ID. NO. 4), gibbon TLR4 polynucleotide (SEQ. ID. NO. 7), rhesus monkey TLR4 polynucleotide (SEQ. ID. NO. 10), capuchin TLR4 polynucleotide (SEQ. ID. NO. 13), squirrel monkey TLR4 polynucleotide (SEQ. ID NO. 16), and baboon TLR4 polynucleotide (SEQ. ID NO. 19).

13. A composition comprising a polypeptide selected from the group consisting of chimpanzee TLR4 polypeptide (SEQ. ID. NO. 3), gorilla TLR4 polypeptide (SEQ. ID. NO. 8), gibbon TLR4 polypeptide (SEQ. ID. NO. 9), rhesus monkey TLR4 polypeptide (SEQ. ID. NO. 12), capuchin TLR4 polypeptide (SEQ. ID. NO. 15), squirrel monkey TLR4 polypeptide (SEQ. ID NO. 18), and baboon TLR 4 polypeptide (SEQ. ID NO. 21).
